(19) United States
(12) Patent Application Publication
(10) Pub. No.: US 2018/0297857 A1
(43) Pub. Date: Oct. 18, 2018

(54) Aqueous Synthesis of Polyhedral "Brick-Like" Iron Oxide Nanoparticles for Hyperthermia and T2 MRI Contrast Enhancement, and for Targeting Endothelial Cells for Therapeutic Delivery

(71) Applicant: KENT STATE UNIVERSITY, Kent, OH (US)

(72) Inventors: Torsten HEGMANN, Kent, OH (US); Matthew WORDEN, Austin, TX (US); Donald W. Miller, Winnipeg (CA)

(21) Appl. No.: 15/578,907

(22) PCT Filed: May 25, 2016
(86) PCT No.: PCT/US16/34046

§ 371 Date: Dec. 1, 2017

Related U.S. Application Data
(60) Provisional application No. 62/169,602, filed on Jun. 2, 2015.

(51) Int. Cl. C01G 49/08; A61K 49/18

(52) U.S. Cl. CPC C01G 49/08; A61K 49/1848; A61K 49/1806; C01P 2004/64; B82Y 40/00; C01P 2002/72; C01P 2004/04; C01P 2006/42; C01P 2004/38

(57) ABSTRACT

A low temperature, aqueous synthesis of polyhedral iron oxide nanoparticles (IONPs) is presented. The modification of the co-precipitation hydrolysis method with Triton X surfactants results in the formation of crystalline polyhedral particles. The particles are herein termed iron oxide “nanobricks” (IONBs), as the varieties of particles made are all variations on a simple “brick-like”, polyhedral shape such as rhombohedral shape or parallelogram as evaluated by TEM. These IONBs can be easily coated with hydrophilic silane ligands, allowing them to be dispersed in aqueous media. The dispersed particles are investigated for potential applications as hyperthermia and T₂ MRI contrast agents. The results demonstrate that the IONBs perform better than comparable spherical IONPs in both applications, and show r₂ values amongst the highest for iron oxide based materials reported in the literature.

### FIELD OF THE INVENTION

A low temperature, aqueous synthesis of polyhedral iron oxide nanoparticles (IONPs) is presented. The modification of the co-precipitation hydrolysis method with Triton X surfactants results in the formation of crystalline polyhedral particles. The particles are herein termed iron oxide “nanobricks” (IONBs), as the varieties of particles made are all variations on a simple “brick-like”, polyhedral shape such as rhombohedral shape or parallelogram as evaluated by TEM. These IONBs can be easily coated with hydrophilic silane ligands, allowing them to be dispersed in aqueous media. The dispersed particles are investigated for potential applications as hyperthermia and T₂ MRI contrast agents. The results demonstrate that the IONBs perform better than comparable spherical IONPs in both applications, and show r₂ values amongst the highest for iron oxide based materials reported in the literature.

### BACKGROUND OF THE INVENTION

Iron oxide nanoparticles (IONPs, composed of either magnetite, Fe₃O₄, or maghemite, Gamma-Fe₂O₃) have been used for several decades in many disparate fields, including environmental remediation, energy storage, and catalysis. In addition, an increasingly prominent area of investigation is in biomedical applications, including drug delivery, MRI contrast enhancement, and magnetic hyperthermia.^{III} The effects of the surface chemistry of functionalized IONPs on such biological and medical applications are fairly well established, as demonstrated in various reviews on the topic. A frequently overlooked aspect of these reviews, however, is the effect that particle morphology may have on these applications. This is unsurprising, as the vast majority of publications deal with very similar core shapes and sizes, almost certainly because most synthetic methods for creating IONPs yield particles of similar morphology (i.e. quasi-spheres in the range of a few to tens of nanometers).

In terms of aqueous syntheses, one of the oldest and most widely used methods is the co-precipitation method by Massart, in which a mixture of Fe²⁺ and Fe³⁺ salts are hydrolyzed under basic conditions in water, yielding roughly spherical IONPs. A more recent method by Yathindranath et al. accomplishes the same by reducing Fe(acac)₃ with NaBH₄ in basic conditions at room temperature. Both of these methods allow for simple coating of the resulting particles by the addition of hydrophilic functionalizing agents such as siloxanes and certain polymers.

In general, non-aqueous methods offer a greater degree of control over particle size and homogeneity. One of the earliest and most widely cited examples, reported by Sun et al., involves Fe(acac)₃ reacted at 265° C. in a mixture of phenyl ether, an alcohol, oleic acid, and oleylamine, with the latter two compounds acting as in situ stabilizing ligands. This method produces highly monodisperse, pure magnetite particles below 20 nm in size. The basic strategy of thermal decomposition of an iron precursor in a high boiling point solvent to create spherical IONPs has been repeated and modified a number of times. Recently, further modifications on this method have allowed for the creation of non-spherical particles. Lee et al. developed a thermal decomposition reaction of Fe(CO)₅ in a mixture of DMF and various imidazolium-based ionic liquids to create Gamma-Fe₂O₃ NPs of various shapes. Depending on the reaction conditions and specific ionic liquid used the particles could be short “bars” about 50 nm in length, or longer “wires” several hundred nanometers in size. Yang et al. synthesized magnetite “nanocubes” by heating Fe(acac)₃ in a mixture of oleic acid, oleylamine, 1,2-hexanediol and benzyl ether. This method allowed them to obtain monodisperse nanocubes as small as 6.5 nm. Guardia et al. demonstrated a somewhat tighter control on the size and distribution of nanocubes at a lower temperature by replacing oleic acid with decanoic acid. Palchudhury et al. performed a relatively low temperature (150° C.) decomposition reaction of iron oleate to create what they termed “iron oxide nanowhiskers” approximately 50 nm long. However, these nanowhiskers are poorly crystalized and must undergo post-synthesis calcination. Bao et al. demonstrated a synthesis of iron oxide nanorods that allowed for some degree of control over size and aspect ratio. A recent publication by Mitra et al. showed that changes in heating rate during a synthesis that normally results in spherical particles resulted in octahedral particles. The authors suggested that the lower surface anisotropy of octahedra, as compared with that of spheres, causes the differences in certain magnetic properties between these types of particles.

### SUMMARY OF THE INVENTION

In almost all cases mentioned above, non-spherical IONPs have been made at high temperature, non-aqueous conditions and often with the use of toxic solvents, which does not directly result in particles that are dispersible in aqueous media. Post-synthesis ligand exchange procedures must be conducted to allow for the particles to be dispersed in biologically relevant media. In order to avoid such extra steps, not to mention the additional costs associated with high temperature methods and organic solvents, an aqueous method that allows for the control and alteration of the IONP morphology and permits simple functionalization with hydrophilic ligands is highly desirable.

An aqueous reduction/hydrolysis of iron(III) chloride with sodium borohydride was conducted in the presence of Triton X100, a surfactant that when mixed with water behaves as a lyotropic liquid crystal (LLC). This synthesis resulted in the formation of 2D sheets composed of a mixture of crystalline iron and iron oxide. These sheets, while only a few nanometers thick, were much larger in other dimensions (hundreds of nanometers) and the total size was difficult to control. Herein we report a modification of that technique which offers more precise control of particles fully “nano” in all dimensions, composed entirely of a single phase of iron oxide. A mixture of iron chloride precursors was hydrolyzed in water with sodium hydroxide in the presence of a surfactant (Triton X45 or Triton X100). The resulting iron oxide particles are a mixture of anisometric polyhedral particles with “brick-like” shapes of varying aspect ratios. The precise ratio between particle types depends on the reaction conditions. The use of Triton X45—which can form a lamellar phase LLC in water—allows for the formation of predominantly rectangular and rhombohedral “nanobricks” labelled IONBsX45. The synthesis with Triton X100—which, in a 50% mixture with water, forms a hexagonal phase LLC—allows for the formation of smaller cubic and rhombohedral particles, labelled IONBsX100. These particles can be easily functionalized with siloxane molecules, allowing for dispersal in aqueous media and thus potential use in biomedical applications.

The present invention relates to the production of crystalline, polyhedral such as rhombohedral or parallelogram iron oxide particles derived by various process steps including dissolving various ferric and ferrous salts in water, heating the formed solution, adding a nonionic surfactant thereto and forming a lyotropic liquid crystal solution that must be at a temperature generally in excess of ambient and will vary depending upon the concentration of the surfactant in water, the types of salts utilized, and the like. Subsequently, an alkylene compound is added to the solution and reacted for sufficient time so that Fe₃O₄ nanoparticles having a non-spherical shape are formed.

More specifically, the various ferric and ferrous salts include halides such as chlorine, bromine, iodine, or other counter ions such as nitrate, sulfate, and acetylacetonate, and the like. Such various ferric and ferrous salts derived from organic ionic species with preferred examples including ferric chloride and ferrous chloride hydrate are placed in water and dissolved. Additional process steps include heating the dissolved composition, and adding a surfactant thereto such as a nonionic surfactant thereto. Suitable heated water temperatures range from about 25° C. to about 80° C., desirably from about 40° C. to about 60° C., and preferably about 50° C. The nonionic surfactants include generally R-phenol-alkoxylates such as ethoxylates where R is an aliphatic such as from about C1 to about C15 and desirably from about C1 to about C8. The ethoxy unit generally contains from about 7 to about 70 and preferably from about 8 to about 40 ethylene oxide repeat units. Such surfactants are well known to the literature and to the art and include various Triton X or various Brij surfactants available from chemical supplies such as Aldrich and, and others, with specific and desired examples including Triton X45® and Triton X100®. Upon slight cooling, a lyotropic solution is formed.

A strong alkaline compound is then added such as sodium hydroxide, potassium hydroxide, or ammonia, and Fe₃O₄ nanoparticles having a non-spherical shape or form, e.g. rhombohedral nanoparticles or parallelogram, are produced having a size from about 3 to about 50 nm, desirably from about 5 to about 30 nm, and preferably from about 7 to about 25 nm. The particles have a negative surface charge over a wide range of millivolts that depends on the silane coating, the pH of the mixture, and the size of the particles. The Zeta potential varies from +50 to −50 mV, and in an EDT silane coating from about −35 to about −45 mV. The d-spacing generally does not vary since it is a feature of the crystal packing and generally has a value of approximately 4.9 angstroms. The molar ratio between the various ferric and ferrous salts is approximately 2.0.

The reduced temperature of the formed bulk lyotropic liquid crystals in the surfactant solution is important and the same needs to be adjusted to maintain the bulk lyotropic liquid crystal phase. These temperatures depend on the type of surfactant used as well as the amount or concentration of the surfactant in the water. Temperatures are generally in excess of ambient and as from about 20° C. or about 25° C. to about 80° C., and desirably from about 25° C. or about 35° C. to about 50° C. It is preferably about 35° C. for Triton X45® and preferably for Triton X100® about 30° C. The amount of the nonionic surfactant based upon 100 parts by weight of water, overall is from 20 to about 60; or about 25 to about 55. It is generally from about 30 to about 60, and preferably from about 40 to about 55 parts by weight for Triton X100®. The amount is from about 20 to about 60, and preferably from about 25 to about 55 parts by weight for Triton X45® and is based upon need depending on the iron chloride compounds.

The amount of the alkaline agents such as sodium hydroxide, potassium hydroxide or ammonium hydroxide is generally adjusted to adjust the pH of the final mixture from about 10 to about 12, and preferably to 11. Reaction time is generally from about 10 minutes to about 2 hours and desirably from about 30 minutes to about 1 hour. The temperature of the alkaline reaction is the same as the formation temperature of the lyotropic liquid crystals.

Two such applications, as noted above, are in the areas of magnetic hyperthermia and MRI contrast enhancement. The former technique involves exposing magnetic particles (either ferro- or superparamagnetic, depending on the material) to an external AC magnetic field, the energy of which is converted to heat in the particles through Brownian and Neel relaxation mechanisms. A number of publications have looked at IONPs in hyperthermia treatment of cancer and tumor cells, as well as infectious agents such as bacteria, both in vivo and in vitro, with promising results. Particle size and shape can play important roles in the overall efficiency of this process. With this in mind we compared both types of NPs, functionalized with a hydrophilic siloxane, with spherical IONPs coated with the same siloxane. The results demonstrate that both kinds of anisometric NPs are significantly more efficient than comparable spherical IONPs for hyperthermia applications.

The superparamagnetic behavior of IONPs allow them to be used as T₂ (negative contrast) agents in MRI diagnostics. Indeed a number of commercial IONP-based contrast agents have been clinically available for many years. All of these are spherical particles, however. As with the properties associated with hyperthermia, particle shape can affect the efficiency of IONPs as MRI contrast agents. We analyzed the same particle systems mentioned above, at varying magnetic field strengths, for their efficacy as T₂ contrast agents. Our anisometric NPs provided both higher r₂ relaxivity values as well as higher r₂/r₁ ratios than comparable spherical NPs, and in fact performed similarly to the most effective commercially available particles currently on market.

### Results and Discussion

Synthesis of IONBsX45 and IONBsX100

In a typical experiment FeCl₃ (2 mmol) and FeCl₂.4H₂O (1 mmol) were dissolved in 20 mL of degassed water and added to a 3 neck round bottom flask under nitrogen. This reaction vessel was heated to 50° C., at which point 25 mL of degassed Triton X surfactant (either X45 or X100, depending on the experiment) was added and mechanically stirred at 100 rpm to ensure that a homogeneous mixture was formed. The vessel was then cooled to 35° C. in the case of X45, and 30° C. in the case of X100. NaOH (30 mmol) was then dissolved in 5 mL degassed water and added to the above mixture under nitrogen and 100 rpm mechanical stirring. The thick, yellow mixture quickly turned black as the NaOH was mixed in. The reaction was left to mix for 1 hour. The black product was washed with warm water and centrifuged at 10,000 rpm several times to isolate it from the surfactant, then dried under nitrogen and stored as a powder under ambient conditions.

Synthesis of S-IONBsX45 and S-IONBsX100

Silanized IONBs (S-IONBs) were synthesized following a modification of the above procedure. One hour after the addition of the NaOH solution, 15 mL of EDTS (45% in water) was added via syringe directly into the reaction vessel. The reaction was left to mix for 12 hours. The product was isolated via multiple washings with a water/ethanol mixture and centrifugation at 10,000 rpm, and then dried under nitrogen. The black powder could then be stored under ambient conditions or re-dispersed in aqueous solution for further use.

### BRIEF DESCRIPTION OF THE DRAWINGS

The invention will be better understood and other features and advantages will become apparent by reading the detailed description of the invention, taken together with the drawings, wherein:

FIG. 1 shows the powder X-ray diffraction (XRD) patterns of bare IONBsX100 and IONBsX45;

FIG. 2A-D shows representative high-resolution (HR)-TEM images of IONBsX45;

FIG. 3A-D shows representative HR-TEM images of IONBsX100;

FIG. 4 shows comparison between a representative polyhedral brick-like shape with how it may appear with changes to size and viewing position. FIG. 4A shows this rhombohedral shape as seen perpendicular to its face, FIG. 4B and FIG. 4C show HR-TEM images of this for IONBsX45 and IONBsX100, respectively. Elongation along one edge leads to a parallelepiped as in FIG. 4D. HR-TEM images of this shape for IONBsX45 are shown in FIG. 4E and FIG. 4F. The same shape as FIG. 4D seen along an edge leads to a purely rectangular shape, as in FIG. 4G. HR-TEM images FIG. 4H and FIG. 4I show examples of this shape seen in IONBsX45.;

FIG. 5 shows visualization of the discussion on the possibility of octahedral vs. rhombohedral shapes. FIGS. 5A and 5B show HR-TEM images of particles from IONBsX45. Similar images can be seen in other syntheses which conclude these are octahedral particles (as in FIG. 5C). FIG. 5D shows how two rhombohedral particles could fuse via mesoscale assembly to form one larger polyhedral particle. FIG. 5E shows a TEM image of these two types of particle shapes side by side from IONBsX45;

FIG. 6 shows magnetic hysteresis curves for IONBsX100 (triangles) and IONBsX45 (squares) at 300 K. Inset shows a magnified image of the coercivity for each particle set;

FIG. 7 (Left): Picture of particle dispersions of S-IONBsX45 and S-IONBsX100. (Right): Schematic representation of the reaction scheme resulting in coated brick-like particles;

FIG. 8 Increase in temperature vs. time for different particle dispersions during exposure to AC magnetic field. Inset: calculated SLP values for each set of particles. AC field was set at an amplitude of 20 kA/m, and a frequency of 2 MHz;

FIG. 9 shows iron oxide nanobricks prepared by co-precipitation in lyotropic liquid crystal phases are versatile and effective theranostic materials for magnetic hyperthermia, T2 MRI contrast enhancement and differential cell internalization;

FIG. 10 shows POM images under crossed polarizers showing birefringent textures of lyotropic phases. A shows an image of a 50% mixture of Triton X45 in water at 35° C.; B shows the same along with a 2:1 mixture of FeCl3 and FeCl2, which represents the condition of the reaction mixture before hydrolysis with Na011 occurs. Fingerprint textures typical of a lamellar phase can be seen in both. C shows an image of a 50% mixture of Triton X100 in water at 30° C.; D shows the same along with a 2:1 mixture of FeCl3 and FeCl2. Focal conic textures typical of a hexagonal phase can be seen in both (limited transmission due to presence of iron salts), although the inclusion of the iron precursors does lower the transition temperature slightly;

FIG. 11A and FIG. 11B show weight-loss vs. temperature profiles of S-IONBsX100 (weight loss) and S-IONBsX45 (˜46% weight loss), respectively; FIG. 11C shows FT-IR spectra of S-IONBsX45 (top) and S-IONBsX100 (bottom). The broad peaks centered at ˜3400 cm-1 corresponds to O—H stretching; the sharp peaks at ˜2915 and ˜2852 cm-1 correspond to C—H stretching the broad peaks at ˜1600 cm-1 are indicative of COO— stretching; the broad peaks centered around ˜1000 cm-1 are due to Si—O—R stretching; and the large asymmetric peak at ˜590 cm-1 correspond to stretching modes associated with Fe2+ —O and Fe3+ —O;

FIG. 12 shows the TEM images showing particles from IONBsX45 at different viewing angles. The top left set shows a typical rhombohedral particle, and the top right shows the same particle when viewed at an alpha (in plane) tilt of negative 20°. The bottom left shows a different rhombohedral particle, and the bottom right shows the same particle when viewed at an alpha (in plane) tilt of positive 20°. Both sets of images demonstrate how changes in viewing angle change the apparent internal angles of the particles and thus affect the evaluation of the particle morphology;

FIG. 13 shows plots used to determine relaxivity values for various particles. y-Axes show the inverse of the relaxation time; x-axes show Fe concentration in mM. A and B are for S-IONBsX100 at 1.5 T and 7 T, respectively; C and D are for S-IONBsX45; E and F are for S-IONPs. Triangle markers correspond to 1/T₂ values; square markers correspond to 1/T₁ values. Equations for determining the slope of each line are included in the inset for each plot;

FIG. 14 shows the TEM images of S-IONBsX100 (A and B), and S-IONBsX45 (C and D).

### EVALUATION OF PARTICLE MORPHOLOGY AND CRYSTALLINITY

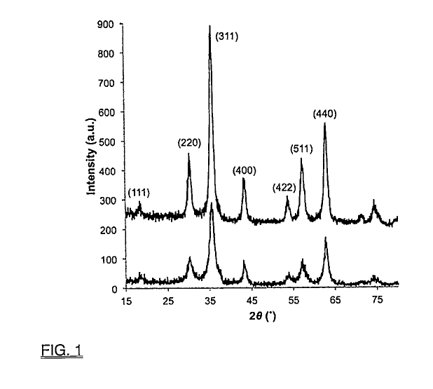

FIG. 1 shows the powder X-ray diffraction (XRD) patterns of bare IONBsX100 and IONBsX45. The indexed patterns match closely to that of bulk magnetite (ICDD reference code 01-089-0691) as well as maghemite.

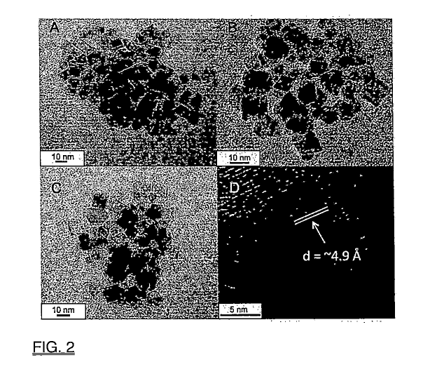

The most important question concerned whether the addition of the surfactants could in fact allow for some measure of control over particle shape. TEM imaging was thus used on the bare IONBsX45 and IONBsX100 in order to answer this question. FIGS. 2A-D shows representative high-resolution (HR)-TEM images of IONBsX45. The particles are composed primarily of rectangular and rhombohedral shapes, with a distribution of sizes of approximately 15 +/−10 nm, and with varying aspect ratios between edge lengths therein. An average d-spacing of approximately 4.9 Å for the visible lattice fringes was determined via analysis with ImageJ® software (FIG. 2D), and is consistent with the d-spacing (4.842 Å) associated with the (111) lattice plane of magnetite.

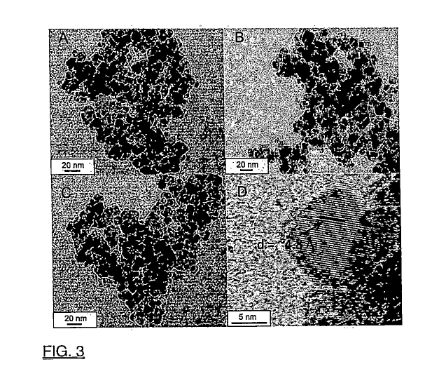

FIGS. 3A-D shows representative HR-TEM images of IONBsX100. These particles differ noticeably from those made with X45. They include primarily rhombohedral shapes with edges of similar lengths of approximately 10 +/−5 nm. The longer rectangular particles seen with X45, in which perpendicular sides have varying aspect ratios, are not seen with X100. An average d-spacing of approximately 4.8 Å was determined (FIG. 3D), again close to that of the d-spacing associated with the (111) lattice plane of magnetite.

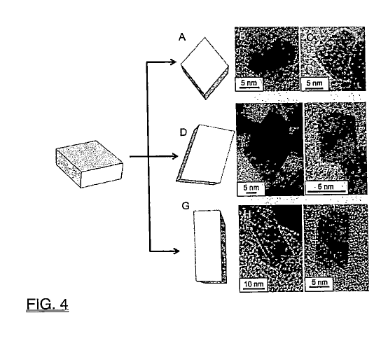

Further evaluation of particle morphology is shown in FIG. 4, which compares TEM images of various particles with 3D representations (shown adjacent to the relevant TEM images) of shapes to which they most likely conform. The figure starts from a rhombohedral “brick”. FIG. 4A shows the face of this rhombohedron, with B (from X45) and C (from X100) showing representative particles with this shape under TEM; FIG. 4D shows a larger shape in which one of the pair of edges is elongated to form a parallelepiped (3D parallelogram), with E and F showing representative TEM images of this shape from IONBsX45; FIG. 4G shows the same shape as D as viewed along the long edge, resulting in a rectangular shape, with H and I showing representative TEM images of this shape from IONBsX45.

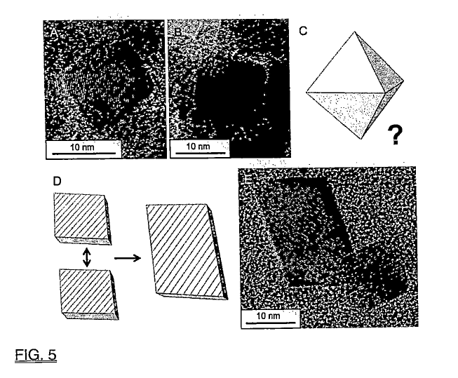

We acknowledge the difficulty in precisely distinguishing between octahedral and rhombohedral shapes based on the 2D information provided by TEM. It is possible that solely one or the other shape is formed exclusively, or even a mixture. FIGS. 5A and 5B show HR-TEM images of IONBsX45 and IONBsX100, respectively, and FIG. 5C shows a 3D representation of an octahedron for comparison.

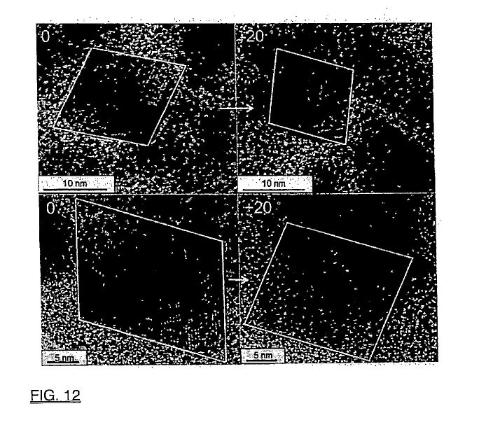

Other researchers, having synthesized IONPs showing similar shape profiles under TEM, have concluded that their particles are octahedral in shape. However, the existence of the larger rectangular and parallelepipedal shaped particles in our synthesis suggests that the rhombohedral shape is more likely. Mesoscale assembly—in which small particles fuse together through alignment of their crystal facets to produce larger particles—is one mechanism through which different particle shapes can be obtained. Octahedral particles could not fuse together to form these kinds of shapes. On the other hand, if two rhombohedral particles fuse together in this manner they would result in a parallelepipedal shape. FIG. 5D shows a schematic representation of this mechanism, and FIG. 5E shows a TEM image of these two types of particles side by side. Examples of images taken with TEM at various tilt angles in order to visualize the effect of sight angle on the apparent shape of a particle are shown in the electronic supplementary information (see FIG. 12, ESI†).

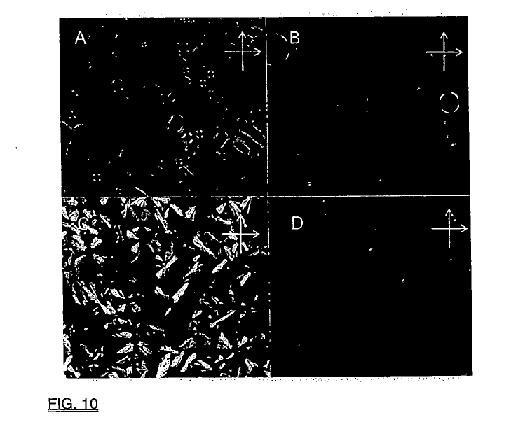

The used Triton X surfactants form LLC phases in water (see FIG. 10 in the ESI† for textures under POM), and, through the formation of these phases, have been used as templates in nanomaterial synthesis. The formation of discrete, layered structures could contribute to the shape control seen here by constraining the direction of growth during particle formation. Other than templating, most shape control in nanomaterials results from control over material growth rate and precursor concentration (i.e. selective access to precursor atoms or monomers through local concentration gradients). In general this is accomplished through some combination of variation in heating rate and the overall temperature of the reaction medium, as well as the use of selective capping agents, which preferentially adsorb onto certain crystal facets. Our modified co-precipitation method requires a much lower reaction temperature than methods using adjusted heating rate and temperature as means of shape control. However, the surfactant molecules may be acting as capping agents. Changes in the nature of capping agent functional groups (i.e. the presence or absence of certain functional groups as well as changes in the ratio between different capping agents) can lead to changes in IONP morphology. Triton X45 and X100 have similar structures, differing only in the length of the ethylene oxide chain. Variations in the chain length of capping agents have been shown to influence shape in other metal oxide particles; the longer PEO chain length of the X100 molecule could account for the restriction in diversity of the resulting particle shapes. The longer X100 chain could also prevent smaller particles from coming close enough together to fuse and form larger particles via mesoscale assembly, which would account for the absence of larger parallelepipedal and rectangular particles in the synthesis of IONBsX100. Given the speed of the hydrolysis reaction and the dynamic nature of the reaction medium, all of the above mechanisms may contribute to the final particle shapes.

### Magnetic Measurements

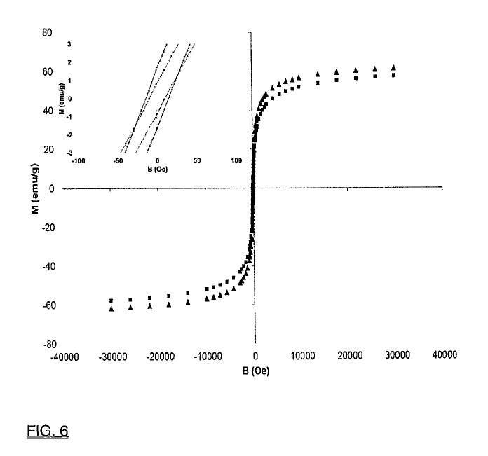

Saturation magnetization (Ms) values were found to be 58 and 61 emu/g at 300 K for IONBsX45 and IONBsX100, respectively. These values are typical of crystalline IONPs on the order of tens of nanometers in size. Both types of particles were found to have low coercivity (Hc) values of 18 and 20 Oe for IONBsX45 and IONBsX100, respectively. These results are shown in FIG. 6.

Surface Functionalization and Characterization

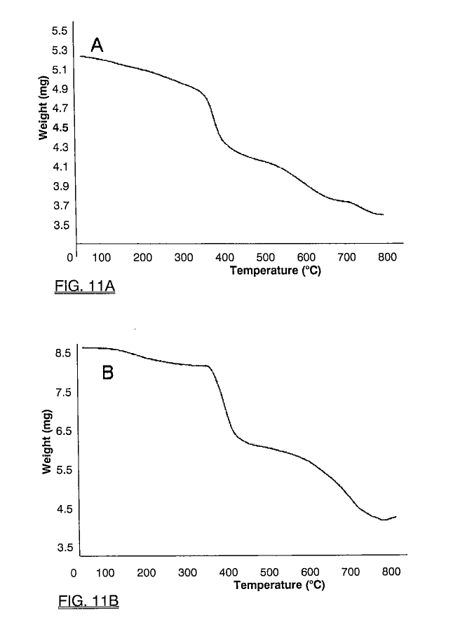

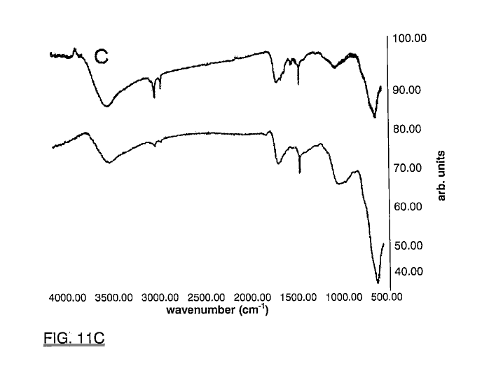

One of the many benefits of an aqueous synthesis is the ease with which particles can be coated with hydrophilic functional groups. In the present case, the IONBs were coated with N-(trimethoxysilylpropyl)ethylenediaminetriacetate trisodium salt (EDTS) through injection of the silane solution directly into the reaction media after the synthesis. Particles coated with the above siloxane are labelled S-IONBsX. EDTS can impart a high negative surface potential on the particles, which allows them to be easily stabilized in aqueous media. Additionally, previous investigations on the cell viability and uptake properties of EDTS-coated spherical IONPs have demonstrated the usefulness of this molecule as a functionalizing agent for IONPs in bioapplications. The dried particles were analyzed by TEM to corroborate the formation of nanobrick shapes as well as FT-IR and TGA to confirm the presence and binding of the EDTS surface coating (see FIGS. 11 and S5, ESI†). The dried particles could be re-suspended in water with mild sonication, and remained stable for weeks without any sign of precipitation.

TABLE 1
Physiochemical properties of silanized particles in water.

| | | ζ-potential | |
|---|---|---|---|
| Material | DLS (nm) | (mV) | pH |
| S-IONBsX45 | 50.9 ± 1.4 | −44.2 ± 2.4 | 9.8 |
| S-IONBsX100 | 64.0 ± 0.4 | −38.1 ± 1.0 | 9.4 |
| S-IONPs | 30.3 ± 1.0 | −36.8 ± 2.8 | 10.2 |

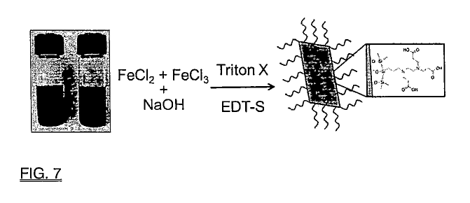

Characterization of the particle suspensions was done with dynamic light scattering (DLS) and ζ-potential measurements. FIG. 7 shows a set of images of 5-IONBsX suspended in water, and a summary of their properties in solution can be found in Table 1. For comparison, quasi-spherical IONPs were synthesized and coated with the same siloxane, following a previous report. These particles are labelled S-IONPs. It should be noted that while the particles were added to distilled water rather than a buffer, the pH of the media was basic due to the nature of the surface coating itself (which contains the conjugate base of a carboxylic acid). Both particle size and surface potential when in solution are highly dependent on pH, as well as ionic strength, which may explain the slight differences between the X45 and X100 particles. These particle dispersions were then used for both MRI relaxivity and hyperthermia measurements described below.

### MRI Relaxivity Measurements

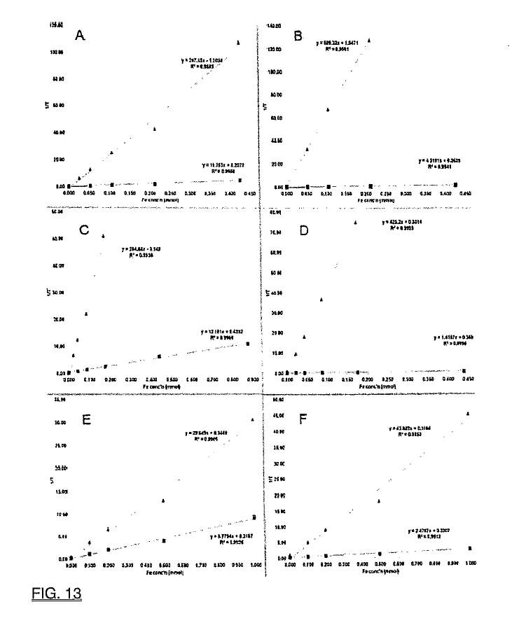

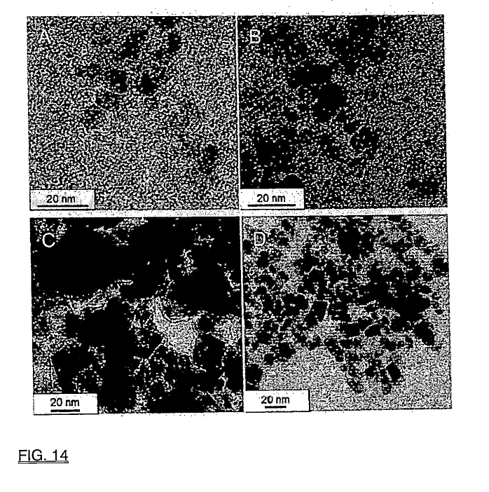

The transverse (r₂) and longitudinal (r₁) relaxivities of S-IONBsX45 and S-IONBsX100 are shown in Table 2 (see FIG. 13 in ESI† for relaxation time vs. Fe concentration plots used to calculate r₂ and r₁). The properties of quasi-spherical 5-IONPs were also measured for comparison. Table 2 lists results at 1.5 T and 7 T. The r₁ values for the three particle types are low and roughly similar. Since iron oxide materials are not typically considered viable T₁ contrast agents, this is unsurprising. The r₂ values of both types of anisometric particle systems are an order of magnitude higher than the spherical IONPs, however. This is true at both 1.5 and 7 T field strengths. The ratio of r₂/r₁—which is a measure of the efficiency of the contrast agent, where a low ratio is favorable for T₁ agents, and a high ratio is favorable for T₂ agents—is also shown. These results demonstrate that the polyhedral NBs are much more efficient T₂ contrast agents as compared with the spherical NPs coated with the same surface ligands.

TABLE 2
Relaxivity values at different field strengths for EDTS coated particles.

| | @ 1.5 T | | | @ 7 T | | |
|---|---|---|---|---|---|---|
| | r₁ | r₂ | | r₁ | r₂ | |
| | (mM⁻¹ s⁻¹) | (mM⁻¹ s⁻¹) | r₂/r₁ | (mM⁻¹ s⁻¹) | (mM⁻¹ s⁻¹) | r₂/r₁ |
| S-IONPs | 8.8 | 29.6 | 3.4 | 2.5 | 43.9 | 17.7 |
| S-IONBs X45 | 12.2 | 285 | 23.4 | 1.4 | 423 | 298 |
| S-IONBs X100 | 11.8 | 247 | 21.0 | 4.3 | 599 | 139 |

Table 3 shows the relaxivity properties of selected iron oxide based contrast agents reported in the literature, both commercial and otherwise. Feridex and Combidex are two types of commercial IONP-based contrast agents, often used for baseline comparison. Their specific properties vary depending upon the literature cited (two examples are given), but in all cases both S-IONB systems show an order of magnitude higher r₂ and r₂/r₁ values.

MPIOs (micrometer-sized iron oxide particles), aggregates of nanometer sized IONPs, have been shown to increase the r₂ value as compared with dispersions of non-aggregated particles. Even still, our polyhedral particles have nearly double the r₂ value seen with MPIOs. Three other types of particles that do show higher r₂ values have been included as well.

FIONs (ferrimagnetic iron oxide nanocubes) have a reported r₂ value of 324 mM⁻¹ s⁻¹ measured at 1.5 T; VNPs (virus-based nanoparticles) have a reported value of 140.28 mM⁻¹ s⁻¹, with a favorable r₂1 r₁ value of 144.6, measured at 3 T; and octapod IONPs have a reported value of 679 mM⁻¹ s⁻¹ measured at 7 T. Each system contains highly anisometric particles, demonstrating the important effect that particle morphology has on their efficiency in MRI applications. In all above cases, particles were synthesized using variations on thermal decomposition methods. We contend that since the relaxivity values reported are not significantly different (324 vs. 285 mM⁻¹ s⁻¹ for FIONs vs. S-IONBsX45; 679 vs. 599 mM⁻¹ s⁻¹ for octapod IONPs vs. S-IONBsX100), the benefits associated with our synthesis allow our particles to be viable alternatives.

TABLE 1
Comparison between commercially available and literature reported
iron oxide based contrast agents.

| | r₂ | | | |
|---|---|---|---|---|
| | (mM⁻¹ | | Field | |
| | s⁻¹) | r₂/r₁ | strength | references |
| Feridex | 41, 98.3 | 8.7, 4.1 | 1.5 T | M. Rohrer, et al., Invest. |
| | | | | Radiol., 2005, 40(11), 715- |
| | | | | 24 and Y. Wang, Quant. |
| | | | | Imaging Med. and Surg., 2011, |
| | | | | 1(1), 35-40. |
| Resovist | 61, 151 | 7.1, 5.9 | 1.5 T | Y. Wang, Quant. Imaging Med. |
| | | | | and Surg., 2011, 1(1), 35-40. |
| | | | | and N. Lee, et al., Proc. Natl. |
| | | | | Acad. Sci. U.S.A., 2011, 108(7), |
| | | | | 2662-2667 |
| MPIO | 169 | — | 1.5 T | N. Lee, et al., Proc. Natl. Acad. Sci. |
| | | | | U.S.A., 2011, 108(7), 2662-2667 |
| FION | 324 | — | 1.5 T | N. Lee, et al., Proc. Natl. Acad. Sci. |
| | | | | U.S.A., 2011, 108(7), 2662-2667. |
| VNPs | 140.28 | 144.6 | 3 T | X. Huang, et al., ACS Nano, 2011, |
| | | | | 5 (5), 4037-4045. |
| Octapod | 679 | — | 7 T | Z. Zhao, et al., Nature Comm., |
| IONPs | | | | 2013, 4, 2266. |

### Hyperthermia Measurements

The hyperthermia performance of the S-IONBs was evaluated by exposing particle dispersions to an AC magnetic field on a custom instrument (described in a previous report). The SLP (specific loss power, also often referred to as SAR, specific absorption rate) values of the samples were calculated in order to measure the efficiency of the particles at converting the magnetic field energy to heat with respect to the amount of iron in each sample. This was calculated using equation (1):

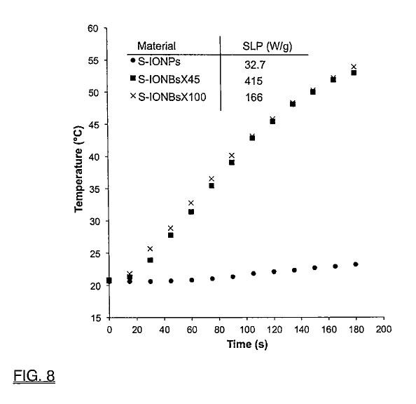

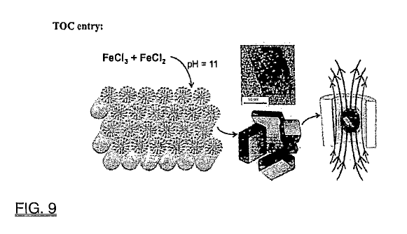

SLP=Cmₛ/m (ΔT/Δt) (1)

where C is the specific heat of the solution (taken to be the same as water, 4.186 J/g° C.), mₛ is the mass of the solution, m is the mass of the magnetic material (in this case the mass of Fe, established by ICP analysis), and ΔT/Δt is the slope of the heating curve. SLP values are highly dependent on the strength of the magnetic field, the nature of the media, how one chooses to evaluate the ΔT/Δt curve, and even the placement of the temperature probe. As such, comparisons between materials used by different researchers with different experimental setups and protocols are problematic. In order to obtain an internal comparison the IONBs were compared with quasi-spherical IONPs to act as a kind of internal standard, as with the MRI relaxivity measurements. FIG. 8 shows the temperature vs. time curves for the three types of particle dispersions measured, along with the calculated SLP values. The graphs clearly show that the quasi-spherical particles (S-IONPs) perform the worst, giving rise to a mere 2.6° C. temperature change over 3 minutes, yielding an SLP value of 32.7 W/g. The IONBs show an order of magnitude more efficient heating response, with a 28.3° C. temperature change and 166 W/g SLP value for S-IONBsX100, and a 29.2° C. temperature change and a 415 W/g SLP value for S-IONBsX45.

Previous reports have suggested that single crystalline particles on the order of 18 nm in size are the most efficient for hyperthermia applications. This may account for the low SLP value for the S-IONPs, which are <10 nm according to the literature. However, the same report suggests that low poly-dispersity in particle size yields greater efficiency. This should preclude higher SLP values for S-IONBsX45 and S-IONBsX100 given their high polydispersity due to the mixture of shapes produced. In the end, the complicated interplay between particle shape, surface anisotropy and overall size distribution may equally contribute to SLP values, which makes direct conclusions as to what accounts for the numbers seen here difficult. Further work in the isolation of particular shapes from the mixtures produced will allow for a better understanding as to which properties contribute the most to high SLP values.

### Conclusions

This study presents the synthesis of polyhedral particles of iron oxide via a modification of the aqueous co-precipitation method with Triton X surfactants. A variety of shapes—variations on cubic and rectangular “brick-like” shapes, deemed IONBs—are formed, with the precise mixture dependent on the surfactant used. The resulting particles are highly crystalline, and their surface properties can easily be modified with the in situ addition of a hydrophilic siloxane. Silanized IONBs remain stable when dispersed in water, allowing for applications in medicine. Their efficacy in two such applications, hyperthermia and MRI contrast, were investigated. Both types of particle systems, S-IONBsX45 and S-IONBsX100, have SLP values an order of magnitude higher than spherical IONPs with the same siloxane coating. Additionally, both “nanobrick” systems show highly favorable MRI T₂ contrast properties, with r₂ values comparable to the highest reported in the literature. We have shown an effective alternative strategy for the control of IONP morphology that is simple, cost-effective, water-based, and also environmentally friendly. In addition, these particles have been investigated for their applications in cell uptake and for their potential as drug delivery vehicles. Recent studies by Sun et al. on S-IONBsX45 have shown that these particles are taken up in endothelial cells at a rate far greater than spherical particles with the same surface coating and reasonably similar hydrodynamic radius and c-potential. This demonstrates that IONP shape modification in general, and the specific shapes found in the particles discussed here, offer a potential means of targeted delivery to specific cells without the need for receptor-ligand interactions.

### Experimental Section

### Materials

Iron(II) chloride tetrahydrate (Reagent Plus, 98%), iron(III) chloride (reagent grade 97%), Triton X100 (laboratory grade), Triton X45, and sodium hydroxide (reagent grade, >98%) were purchased from Sigma-Aldrich. N-(trimethoxysilylpropyl) ethylenediaminetriacetate trisodium salt (45% in water) was purchased from Gelest Inc.

Transmission Electron Microscopy (TEM) Imaging

TEM imaging was done with a FEI Tecnai TF20 TEM instrument at an accelerating voltage of 200 kV. Particle samples were dispersed in methanol and dropcast onto 400 mesh carbon coated copper grids.

### XRD Analysis

Powder X-ray diffraction patterns (XRD) were measured on an X′Pert PRO diffractometer manufactured by PANalytical, Inc. (Westborough, Mass., USA). The experimental setup used Bragg-Brentano geometry in θ-θ configuration, copper as a radiation source (Cu Kα radiation), and a diffracted beam curved crystal monochromator to eliminate Cu Kβ. All patterns were collected in a range of 2θ values from 10.00° to 80.00° with a step size 0.05°.

### MRI relaxivity measurements

The ionic relaxivity of the iron oxide particles was tested using a pre-clinical 7.0 T (300 MHz) MRI (Bruker BioSpec 70/30 USR), and a Bruker Minispec mq60 relaxometer (60 MHz). A standard inversion recovery sequence protocol was used to determine the longitudinal T₁ values on each of the instruments. The transverse relaxivity (r₂) of the particles was calculated as the slope of 1/T₂) against iron concentration. T₂ relaxation times were determined using a standard Carr-Purcell-Meiboom-Gill spin echo sequence.

### Hyperthermia Measurements

Dispersions of S-IONBsX45 and S-IONBsX100 were made by sonicating dried powder in deionized water (at concentrations of 9.88 and 9.95 mg/mL, respectively). A dispersion of S-IONPs, synthesized similarly to a previous report, was also made in this manner at a concentration of (10.01 mg/mL). In a typical hyperthermia experiment, 250 μL of particle dispersion was added to a single well from a 96-well plate. The samples were exposed to a field with an amplitude of 20 kA/m and a frequency of 2.1 MHz for 3 minutes while the temperature of the sample media was monitored using a fiber optic temperature probe (Neoptix).

Dynamic light scattering (DLS) and ζ-potential measurements

The hydrodynamic radius and ζ-potential of the S-IONBs were determined using a Brookhaven Zetaplus ζ-potential-DLS measurement system. The instrument specifications include a 35 mW class 1 laser at 660 nm with a scattering angle of 90°. All dispersions were measured at a concentration of ≠1 mg/mL. Results listed are an average of 3 consecutive measurements.

### Polarized Optical Microscopy (POM) Imaging

POM images were taken with an Olympus BX-53 equipped with a Linkam LTS420E heating/cooling stage.

### FT-IR Sample Preparation

Surface functionalization was analyzed through FT-IR using KBr pellet techniques. Approximately 1 mg of dried particles were mixed with approximately 150 mg of KBr, which was then pressed into a pellet. The pellet was stored in a vacuum oven at 50° C. for several hours before analysis to remove any adsorbed water. Spectra were recorded using a Magna Nicolet-500 series FT-IR spectrometer.

### Thermal Gravimetric Analysis (TGA) Measurements

The amount of surface ligands on the particles was estimated via a TA instruments TGA Q500. The heating rate was set at 10° C./min. Powdered samples were typically dried in a vacuum oven at 50° C. for 2 hours before analysis in order to eliminate any surface water.

### Magnetic Measurements

The magnetic properties were characterized with an RF Superconducting Quantum Interference Device (SQUID) magnetometer (Quantum Design MPMS-XL) with reciprocating sample transport. The field was applied between −30 to +30 kOe at 300 K.

While in accordance with the patent statutes the best mode and preferred embodiment have been set forth, the scope of the invention is not limited thereto, but rather by the scope of the attached claims.

What is claimed is:

1. Crystalline iron oxide nanoparticles, comprising:
Fe₃O₄ particles having a non-spherical, polyhedral, (brick-like) shape and a size of from about 3 to about 50 nm.

2. The crystalline iron oxide nanoparticles of claim 1, wherein said particles have a Zeta potential of from +50 to −50 mV.

3. The crystalline iron oxide nanoparticles of claim 2, wherein said particle size ranges from about 5 to about 30 nm, and wherein said particles have a d-spacing of approximately 4.9 angstroms.

4. The crystalline iron oxide nanoparticles of claim 3, wherein said particles have a siloxane coating, and wherein said Zeta value is from about −35 to about −45 mV.

5. The crystalline iron oxide nanoparticles of claim 3, wherein said particle shape is a parallelogram or a rhombohedral.

6. The crystalline iron oxide nanoparticles of claim 4, wherein said particle shape is a parallelogram or a rhombohedral.

7. A method of making crystalline iron oxide particles comprising the steps of:
dissolving ferric salt and ferrous salt in water and forming a mixture, heating said mixture from about 25° C. to about 80° C. and forming a lyotropic liquid crystal phase or micellar solution by adding an ionic surfactant thereto and forming a homogeneous mixture.

8. The method of claim 7, wherein said ferric salt comprises a ferric halide, a ferric nitrate, a ferric sulfate, or a ferric acetylacetonate, or any combination thereof, and wherein said ferrous salt comprises a ferrous halide, a ferrous nitrate, a ferrous sulfate, or a ferrous acetylacetonate, or any combination thereof, and wherein said ionic surfactant has the formula of R-phenyl-O-(ethoxy), wherein n is from about 7 to about 70, and where R is an aliphatic having from 1 to about 15 carbon atoms.

9. The method of claim 8, wherein the amount of said nonionic surfactant is from about 20 to about 60 parts by weight per 100 parts by weight of water; and wherein the mole ratio of said ferric salts to said ferrous salts is about 2.

10. The method of claim 9, wherein the amount of said surfactants is from about 25 to about 55 parts by weight per every 100 parts by weight of said water, wherein said ferric salt is ferric chloride and wherein said ferrous salt is ferrous chloride hydrate, and wherein said ionic surfactant is octylphenyl ethoxate wherein n is 9 or 10, or octylphenyl ethoxate where n is about 40.

11. The method of claim 7, including adding a strong alkaline compound to said lyotropic mixture and forming Fe₃O₄ nanoparticles.

12. The method of claim 10, including adding a strong alkaline compound to said lyotropic mixture and forming Fe₃O₄ nanoparticles.

13. The method of claim 11, wherein said nanoparticles have a size of from about 3 to about 50 nanometers, and wherein said alkaline compound is sodium hydroxide, potassium hydroxide, or ammonium hydroxide, or any combination thereof.

14. The method of claim 13, wherein said nanoparticle size is from about 5 to about 30 nanometers.

15. An MRI contrast agent comprising the composition of claim 9.

16. An MRI contrast agent comprising the composition of claim 14.

17. A hypothermia compound comprising the composition of claim 9.

18. A hypothermia compound comprising the composition of claim 13.
